(19) United States
(12) Patent Application Publication
(10) Pub. No.: US 2024/0400612 A1
(43) Pub. Date: Dec. 5, 2024

(54) AN IMPROVED PROCESS FOR PURIFICATION OF FUSION PROTEIN

(71) Applicant: Kashiv BioSciences, LLC, Piscataway, NJ (US)

(72) Inventors: Om Narayan, Ahmedabad (IN); Mayankkumar Thakkar, Ahmedabad (IN); Anuj Kumar, Ranchi (IN)

(21) Appl. No.: 18/696,701

(22) PCT Filed: Sep. 28, 2022
(86) PCT No.: PCT/IB2022/059240

§ 371 Date: Mar. 28, 2024

(30) Foreign Application Priority Data
Sep. 28, 2021 (IN) 202121043969

(51) Int. Cl. C07K 1/22; A61K 38/00; B01D 15/16; B01D 15/32; B01D 15/36; B01D 15/38; C07K 1/18; C07K 1/20; C07K 14/705

(52) U.S. Cl. CPC C07K 1/22; B01D 15/166; B01D 15/327; B01D 15/363; B01D 15/3809; C07K 1/18; C07K 1/20; C07K 14/70521; A61K 38/00; C07K 2317/52; C07K 2319/30

(57) ABSTRACT

The present invention relates to purification process of pharmacologically active cytotoxic T lymphocyte-associated 4-immunoglobulin (CTLA4-lg) fusion protein by using three column chromatography steps that provides purified form of the fusion protein, substantially free of misfolded species, charge variants and aggregates. A method for purifying a fusion protein from a protein mixture comprising a fusion protein and high molecular weight (HMW) impurity.

### FIELD OF THE INVENTION

The present invention relates to purification process of pharmacologically active fusion protein by using three column chromatography steps arranged in order of Affinity chromatography, Hydrophobic interaction chromatography and Anion exchange chromatography that provides purified form of the fusion protein, substantially free of misfolded species Pre-Peak, product, and process related impurities.

### BACKGROUND OF THE INVENTION

Purification of fusion protein is an increasingly important consideration for biopharmaceutical industries.

With the advancement of biotechnology, fusion proteins are considered a promising therapeutic area for the treatment of diseases. However, fusion proteins are complex in nature as made of fusion of receptor (natural or modified) and immunoglobulin constant region (Fc including with or without hinge region or modified Fc). As per the complexity of the fusion proteins and its challenging purification process gives a motivation to minimize the impurities from fusion protein that proportionally affect the stability and functional efficacy. The recent advances in mammalian cell culture processes have significantly increased product titers as well as process and product-related impurities. Aggregation, charge variants, high molecular weight (HMW), low molecular weight (LMW) like impurities with the fusion protein has been a major problem that has been associated with a change in protein structure and being a hurdle in various upstream and downstream purification processes. The Fc-fusion proteins have elevated levels of aggregates, high molecular weight species (HMWs; up to 20%) and low molecular weight species (LMWs; up to 20%) within the product species. Glycosylation of proteins and the subsequent processing of the added carbohydrates can affect protein folding and structure, protein stability, including protein half-life, and functional properties of a protein. Desired glycosylation can be obtained through clone and upstream process. These impurities generated during the manufacturing process leads to decreased product yield, peak broadening, and reduce or loss of activity of molecule. The improvement or a step forward in Fc-fusion protein processing is crucially needed as conventional chromatography column requires various conditioning parameters of protein before loading onto chromatography column, lacking effective purification process to remove product and process related impurities, that directly impacts process time, cost, and labor effort in protein handling.

The present invention provides a purification process by selecting and arranging the affinity chromatography, hydrophobic interaction chromatography and optionally one or more suitable purification steps to remove impurities associated with the fusion protein. The process reduces or removes at least one or more impurities selected from Pre-Peak, HMW, LMW, HCP, HCD, charge variants and undesired glycan.

There is a present need for methods of producing and purifying a fusion protein of interest in sufficiently pure form to be suitable for pharmaceutical use.

### SUMMARY OF THE INVENTION

In an embodiment, the invention provides a chromatography process to produce a composition enriched with fusion protein of interest and substantially reduced impurities selected from acidic variants, Pre-peak, HMW or aggregates.

In an embodiment, the invention provides a chromatography process to produce a composition enriched with fusion protein of interest and substantially reduced high molecular weight (HMW) impurities.

In an embodiment, the invention provides a chromatography process to produce a composition enriched with fusion protein of interest and substantially reduced pre-peak impurities.

In an embodiment the invention provides a chromatography process to produce a composition enriched with fusion protein of interest and substantially reduced low molecular weight (LMW) impurities.

In an embodiment, the invention is related to the process for the purification of the fusion protein which is free from at least one impurity selected from Host cell proteins (HCP), Host cell DNA (HCD), Low molecular weight (LMW) and High molecular weight (HMW) impurities comprising:
a) obtaining first protein mixture from the suitable mammalian expression system comprising fusion protein and impurities;
b) applying the first protein mixture to affinity chromatography column;
c) eluting the fusion protein from affinity chromatography column wherein the eluted fusion protein is present in second protein mixture contains reduced amount of said impurities;
d) applying the second protein mixture to hydrophobic interaction chromatography column;
e) eluting the third fusion protein from a hydrophobic interaction chromatography column wherein the eluted fusion protein comprises reduce amount of HMW and LMW impurities;
f) applying the third protein mixture to anion exchange chromatography column;
g) eluting the fusion protein from anion exchange chromatography column;

wherein the eluted fusion protein as fourth protein mixture is substantially free of LMW and HMW impurities.

In an embodiment, the invention provides a hydrophobic interaction chromatography and anion exchange chromatography to produce a composition comprising enriched fusion protein of interest and significantly reduced high molecular weight (HMW) impurities selected from about 10%, about 9%, about 8%, about 7%, about 6%, about 5%, about 4%, about 3%, about 2%, about 1%, about 0.5%, or less HMW.

In one aspect of this embodiment, the low HMW composition comprises about 0.5% or less HMW, about 0.4% or less HMW, about 0.3% or less HMW, about 0.2% or less HMW, about 0.1% or less HMW.

In an embodiment, the invention provides an anion exchange chromatography and hydrophobic interaction chromatography to produce a composition comprising enriched fusion protein of interest and reduced LMW impurities selected from about 50%, about 40%, about 30%, about 20%, about 10%, about 9%, about 8%, about 7%, about 6%, about 5%, about 4%, about 3%, about 2%, about 1%, about 0.5%, or less LMW.

In an embodiment, the invention is related to the purification of CTLA-4 IgG1 fusion protein from a protein mixture comprising:
a) obtaining first protein mixture from the suitable mammalian expression system comprising CTLA-4 IgG1 fusion protein and impurities;
b) applying the first protein mixture to affinity chromatography column;
c) eluting the fusion protein from affinity chromatography column wherein the eluted CTLA-4 IgG1 fusion protein is present in second protein mixture contains reduced amount of impurities;
d) applying the second protein mixture to hydrophobic interaction chromatography column;
e) eluting the CTLA-4 IgG1 fusion protein from hydrophobic interaction chromatography column wherein the eluted CTLA-4 IgG1 fusion protein as third protein mixture comprises reduced amount of HMW impurities;
f) applying the third protein mixture to anion exchange chromatography column;
g) eluting the fusion protein from anion exchange chromatography column;

wherein the eluted fusion protein as fourth protein mixture is substantially free of LMW and HMW impurities.

In an embodiment, the invention provides an anion exchange chromatography and hydrophobic interaction chromatography to produce a composition comprising enriched CTLA-4 IgG1 fusion protein and significantly reduced high molecular weight (HMW) impurities selected from about 10%, about 9%, about 8%, about 7%, about 6%, about 5%, about 4%, about 3%, about 2%, about 1%, about 0.5% or 0.3%, or less HMW.

In one aspect of this embodiment, the low HMW composition comprises about 0.9% or less HMW, about 0.8% or less HMW, about 0.7% or less HMW, about 0.6% or less HMW, about 0.5% or less HMW, about 0.4% or less HMW, about 0.3% or less HMW, about 0.2% or less HMW, about 0.1%, or less HMW.

In an embodiment, the invention is related to the purification of CTLA-4 IgG1 fusion protein from a protein mixture comprising:
a) obtaining first protein mixture from the suitable mammalian expression system comprising CTLA-4 IgG1 fusion protein and impurities;
b) applying the first protein mixture to affinity chromatography column;
c) eluting the fusion protein from affinity chromatography column wherein the eluted CTLA-4 IgG1 fusion protein is present in second protein mixture contains reduced amount of impurities;
d) applying the second protein mixture to hydrophobic interaction chromatography column;
e) eluting the CTLA-4 IgG1 fusion protein from hydrophobic interaction chromatography column wherein the eluted CTLA-4 IgG1 fusion protein as third protein mixture comprises reduced amount of reduce pre-peak impurities;
f) applying the third protein mixture to anion exchange chromatography column;
g) eluting the fusion protein from anion exchange chromatography column

wherein the eluted fusion protein as fourth protein mixture is substantially free of pre-peak impurities.

In one aspect of such embodiment, the pre-peak impurity reduced by at least more than 90%, 91%, 92%, 93%, 94%, 95%, 96%, 97%, 98%, 99%. In an embodiment the pre-peak impurity is below quantifiable limit.

In an embodiment, the fusion protein comprises purity of monomer selected from about 96% or more, about 97% or more, 98% or more and 99% or more.

### BRIEF DESCRIPTION OF DRAWINGS

FIG. 1: Depicts the complete chromatogram of the Affinity chromatography run.

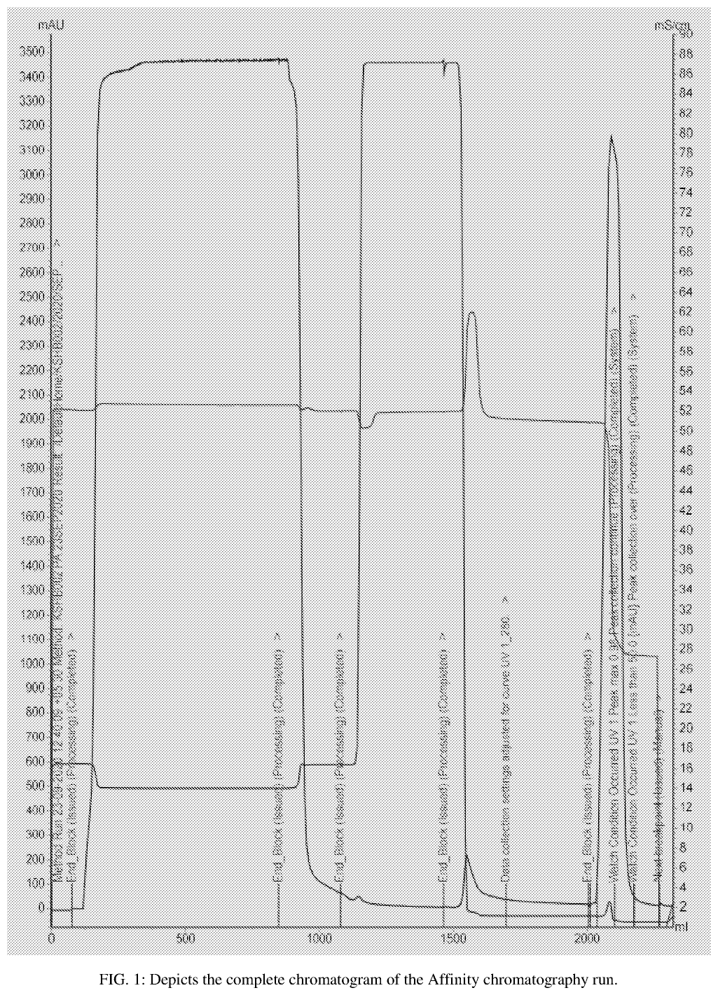

FIG. 2: Depicts the complete chromatogram of the Hydrophobic interaction chromatography run.

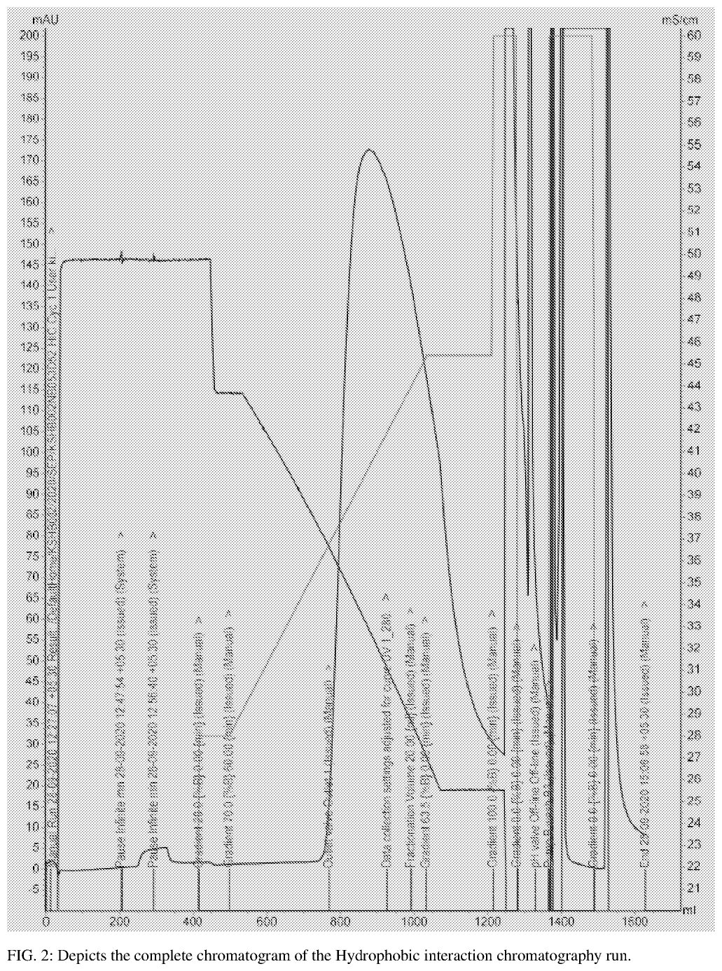

FIG. 3: Depicts the complete chromatogram of the Anion Exchange chromatography run.

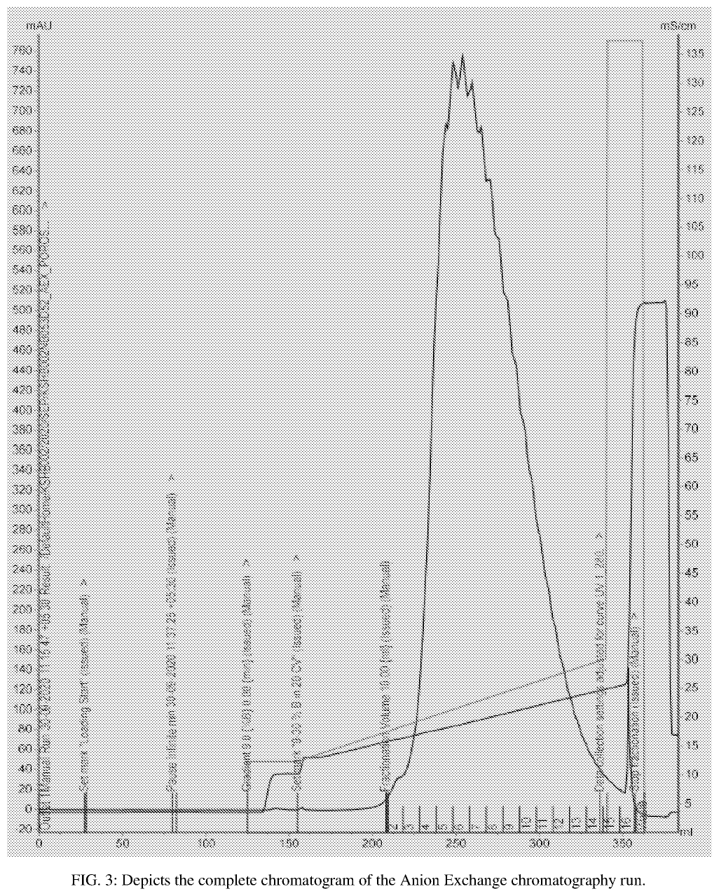

FIG. 4: Depicts SE-HPLC Chromatogram of Final DS

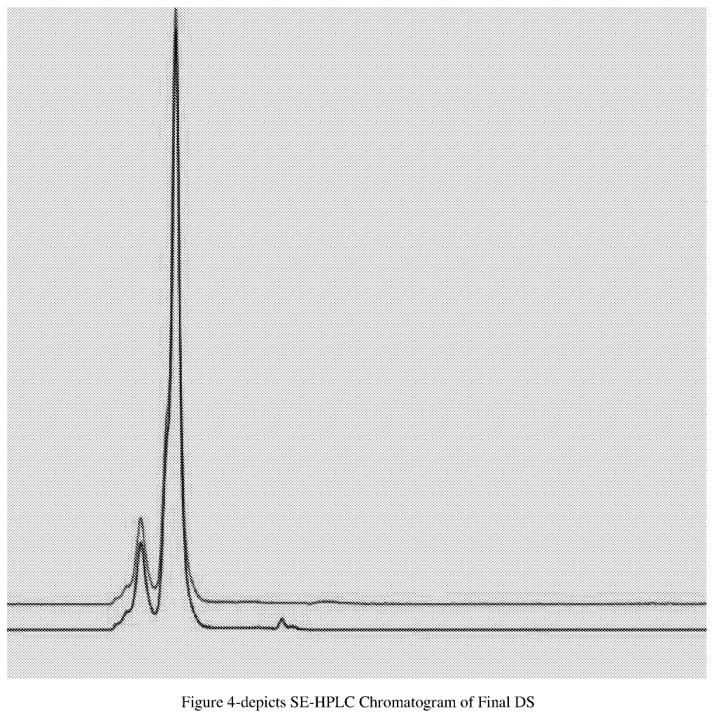

### DETAIL DESCRIPTION OF INVENTION

As used herein the term “column” or “resin” or “chromatographic resin or chromatographic column” are interchangeable.

The term “comprises” or “comprising” is used in the present description, it does not exclude other elements or steps. For purpose of the present invention, the term “consisting of” is considered to be an optional embodiment, of the term “comprising of”. If hereinafter a group is defined to comprise at least a certain number of embodiments, this is also to be understood to disclose a group which optionally consists only of these embodiments. As used throughout the specification and in the appended claims, the singular forms “a,” “an,” and “the” include the plural reference unless the context clearly dictates otherwise.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The term “clarified harvest cell culture fluid” or “HCCF” or “harvest cell culture fluid” used herein are interchangeable refers to the protein mixture obtained from mammalian cell culture containing protein of interest along with other impurities.

### Affinity Chromatography

The term “affinity chromatography” refers to chromatography processes of separating biochemical mixtures based on a highly specific interaction between e.g., antigen and antibody, enzyme and substrate, receptor and ligand, or protein and nucleic acid. Examples of such chromatographic resins include, but are not limited to Protein A resin, Protein G resin, Protein L resin, immobilized metal ion affinity chromatography etc.

The term “Protein A affinity chromatography” use for the separation or purification of substances and/or particles using protein A, where the protein A is generally immobilized on a solid phase. Protein A is a 40-60 kD cell wall protein originally found in Staphylococcus aureus. The binding of fusion protein to protein A resin is highly specific. Protein A affinity chromatography columns for use in protein A affinity chromatography herein include, but are not limited to, Protein A immobilized on a polyvinyl ether solid phase, e.g., the Eshmuno® columns (Merck, Darmstadt, Germany), Protein A immobilized on a pore glass matrix, e.g., the ProSep® columns (Merck, Darmstadt, Germany) Protein A immobilized on an agarose solid phase, for instance the MABSELECT™ SuRe™ columns (GE Healthcare, Uppsala, Sweden).

The term “MabSelect SuRe LX” used herein is a protein A affinity resin with high dynamic binding capacity at extended residence times. The ligand is alkali-stabilized protein A-derived (E. coli) shows alkali tolerance, high capacity and low ligand leakage in combination with the rigid base matrix.

### Hydrophobic Interaction Chromatography (HIC)

The term “hydrophobic interaction chromatography” or “HIC” refers to a column containing a stationary phase or resin and a mobile or solution phase in which the hydrophobic interaction between a protein and hydrophobic groups on the matrix serves as the basis for separating a protein from impurities including fragments and aggregates of the subject protein of interest, other proteins or other protein fragments and other contaminants such as cell debris, or residual impurities from other purification steps. The stationary phase comprises a base matrix or support such as a cross-linked agarose, silica or synthetic copolymer material to which hydrophobic ligands are attached. HIC relies on separation of proteins on the basis of hydrophobic interactions between non-polar regions on the surface of proteins and insoluble, immobilized hydrophobic groups on the matrix. The absorption increases with high salt concentration in the mobile phase and the elution is achieved by decreasing the salt concentration of the eluant.

The term “Poros Benzyl” used herein is Hydrophobic Interaction Chromatography (HIC) resin engineered for improved impurity removal during downstream purification of biomolecules and its capacity maintains excellent pressure flow characteristics, helps increase in productivity. The resin has POROS base bead with aromatic hydrophobic benzyl ligand, suited for purification.

### Anion Exchange Chromatography (AEX)

The term “anion exchange chromatography” or “anion exchange column” or “AEX” used herein is a form of ion exchange chromatography (IEX), which is used to separate molecules based on their net surface charge. Anion exchange chromatography, more specifically, uses a positively charged ion exchange resin with an affinity for molecules having net negative surface charges. Anion exchange chromatography is used both for preparative and analytical purposes and can separate a large range of molecules, from amino acids and nucleotides to large proteins.

The term “POROS XQ” used herein is a Thermo Scientific POROS XQ Strong Anion Exchange Resin are designed for charge based chromatographic separations of biomolecules including recombinant proteins or fusion proteins. POROS XQ has high and consistent protein binding capacity across a broad range of salt concentrations. The resin has quaternary amine groups.

As used herein the term “bind and elute mode” or “B/E” refers to purification process wherein the fusion protein of interest binds to chromatography resin. In certain embodiment, at least 90% fusion protein of interest bind to chromatographic resin. In certain embodiment, at least 60% or 70% or 80% fusion protein of interest binds to chromatographic resin. However, process and product related impurities does not bind the chromatographic resin. In certain embodiment, at least 50% process and product related impurities does not bind to chromatographic resin. In certain embodiment, at least 60% or 70% or 80% process and product related impurities does not bind to chromatographic resin.

The term “Host cell protein” or “HCP” refers to process-related protein impurities produced by the host organism during biotherapeutic manufacturing and production. These HCPs are removed from the final product (>99% of impurities removed).

The term “Host cell DNA” or “HCD” refers to residual DNAs (rDNAs) are trace/low quantity of DNA originating from the organisms used in the production process of biopharmaceutical products, which may be introduced into the final products.

The term “viral reduction/inactivation”, as used herein, is intended to refer to a decrease in the number of viral particles in a particular sample (“reduction”), as well as a decrease in the activity, for example, but not limited to, the infectivity or ability to replicate, of viral particles in a particular sample (“inactivation”).

### Pre-Peak

The term used “pre-peak” demonstrates the analysis of the product related variant. The peak with the shortest retention time arising just before the main peak or monomer which contains product and/or process related impurities.

### Buffer

The term “buffer” or “suitable buffered” refers to solutions which resist changes in pH by the action of its conjugate acid-base range. Examples of buffers that control pH at ranges of about pH 5 to about pH 8 include Tris, citrate, phosphate, sulphate and acetate, and other mineral acid or organic acid buffers, and combinations of these. Salt cations include sodium, ammonium, and potassium. The “loading buffer” or “equilibrium buffer” or “washing buffer” refers to the buffer containing the salt or salts which is mixed with the protein preparation for loading the protein preparation onto the column. This buffer is also used to equilibrate the column before loading, and to wash to column after loading the protein. The “elution buffer” refers to the buffer used to elute the protein from the column.

The term used herein “Buffer B” or “Elution buffer B” in hydrophobic interaction chromatography are interchangeable refers without limiting to the buffer solution of 10 mM TrisAc, pH 8.0±0.2

The term used herein “Buffer B” or “Elution buffer B” in anion exchange chromatography are interchangeable refers without limiting to the buffer solution of 50 mM Tris acetate and 1M Sodium chloride, pH 7.5±0.2.

The term “Neutralized Protein A Elute or NPEL” both are interchangeable and it refers to the removal of protein of interest from the column along with buffer.

The term “neutralization” used herein refers to bringing back the acidic pH of the protein A eluate to a neutral pH by using basic solution.

### Aggregates

The term used “aggregates” are classified based on types of interactions and solubility. Soluble aggregates are invisible particles and cannot be removed with a filter. Insoluble aggregates can be removed by filtration and are often visible to the human eye. Both types of aggregates cause problems in biopharma development. Covalent aggregates arise from the formation of a covalent bond between multiple monomers of a given peptide. Disulphide bond formation of free thiols is a common mechanism for covalent aggregation. Oxidation of tyrosine residues can lead to formation of bityrosine which often results in aggregation. Reversible protein aggregation typically results from weaker protein interactions they include dimers, trimers, multimers among others.

The term “Host cell protein” or “HCP” refers to process-related protein impurities produced by the host organism during biotherapeutic manufacturing and production.

The term “Host cell DNA” or “HCD” refers to residual DNAs (rDNAs) are trace/low quantity of DNA originating from the organisms used in the production process of biopharmaceutical products.

### High Molecular Weight (HMW)

The term used “high molecular weight” or “HMW” is product-related impurities that contribute to the size heterogeneity of fusion protein. The formation of HMW species within a therapeutic fusion protein drug product as a result of protein aggregation can potentially compromise both drug efficacy and safety (e.g., eliciting unwanted immunogenic response). HMW is considered critical quality attribute that are routinely monitored during drug development and as part of release testing of purified drug product during manufacturing. In certain embodiment the HMW relates to aggregates.

### Low Molecular Weight (LMW)

The term used “low molecular weight” or “LMW” species which is a protein backbone-truncated fragments and considered as product-related impurities that contribute to the size heterogeneity of fusion protein. LMW species often have low or substantially reduced activity relative to the monomeric form of the fusion protein and can lead to immunogenicity or potentially impact pharmacokinetic properties in vivo. As a result, LMW species are considered critical quality attributes that are routinely monitored during drug development and as part of release testing of purified drug product during manufacturing.

The term “substantially pure fusion protein” used herein includes a fusion protein that is substantially free of impurity selected from product or process related impurity. The fusion protein is free of pre-peak and high molecular weight, substantially pure fusion protein has purity less than about 99% or less than about 98% or less than about 97% or less than about 95% or less than about 92% or less than about 90% or less than about 88% or less than about 85% or less than about 82% less than about 80% or less than about 75% or less than about 70% or less than about 65% or less than about 60% or less than 50%.

The term “protein mixture” used herein refers to the solutions containing protein of interest along with other impurities, may be or may not be of any chromatography eluate.

The term “first protein sample” or “first protein mixture” or “first sample” used herein is crude protein sample obtained from mammalian cells.

The term “second protein sample” or “second protein mixture” or “second sample” used herein is an affinity chromatography output.

The term “third protein sample” or “third protein mixture” or “third sample” used herein is a hydrophobic interaction chromatography output (HIC OP).

The term “fourth protein sample” or “fourth protein mixture” or “fourth sample” used herein is anion exchange chromatography output (AEX OP).

The term “Residence time” refers to the amount of time a compound spends on the column after it has been injected. If a sample containing several compounds, each compound in the sample will spend a different amount of time on the column according to its chemical composition i.e. each will have a different retention time. Retention times are usually quoted in units of seconds or minutes.

The term “Drug Substance” or “DS” refers to an active ingredient intended to furnish pharmacological activity. The DS also refers to final protein mixture after all purification steps and substantially free from impurities.

In an embodiment, the present invention provides a combination of chromatographic methods selected from protein A chromatography, hydrophobic interaction chromatography, and anion exchange chromatography to purify at least one impurity present in the mixture of CTLA-4 IgG1 fusion protein.

In an embodiment, the invention is related to a process for the purification of fusion protein by using Protein A (ProA) chromatography works in the bind and elute mode.

In another embodiment, the invention is related to a process for the purification of fusion protein followed by Hydrophobic interaction chromatography (HIC) works in the bind and elute mode.

In another embodiment, the invention is related to a process for the purification of fusion protein followed by Anion exchange chromatography (AEX) works in the bind and elute mode.

In yet another embodiment, the invention is related to the purification of fusion protein mixture comprising:
a) loading the fusion protein mixture onto first chromatography column;
b) optionally performing the washing;
c) eluting the fusion protein mixture from said first chromatography column;
d) optionally performing the filtration;
e) loading the fusion protein mixture obtained from step (C) or (D) onto hydrophobic interaction chromatography;
f) optionally performing the washing;
g) eluting the fusion protein mixture from said hydrophobic interaction chromatography column;

wherein the first chromatography column is selected from Protein-A column, Hydrophobic interaction chromatography, Anion exchange chromatography and Cation exchange chromatography.

In yet another embodiment, the invention is related to the purification of fusion protein by performing protein A chromatography, which is followed by hydrophobic interaction chromatography (HIC), which is followed by anion exchange chromatography.

In another embodiment, the invention is related to the purification of fusion protein by performing protein A chromatography, which is followed by hydrophobic interaction chromatography (HIC), optionally further comprises one chromatography step.

In such embodiment the virus inactivation is not performed between protein A chromatography and HIC. In one aspect of such embodiment, pH-based virus inactivation is not performed in eluate of affinity chromatography.

In an embodiment, the invention is related to the purification of fusion protein by performing protein A chromatography, followed by optional viral inactivation or removal, which is followed by hydrophobic interaction chromatography (HIC), optionally further comprises one chromatography step.

In an embodiment, the invention is related to the purification of fusion protein by performing protein A chromatography, followed by hydrophobic interaction chromatography (HIC), which is followed by optional viral inactivation or removal, optionally further comprises one or more chromatography step.

In an embodiment, the invention is related to the purification of CTLA-4 IgG1 fusion protein from a protein mixture comprising:
a) obtaining first protein mixture from the suitable mammalian expression system comprising CTLA-4 IgG1 fusion protein and impurities;
b) applying the first protein mixture to affinity chromatography column;
c) eluting the fusion protein from affinity chromatography column wherein the eluted CTLA-4 IgG1 fusion protein is present in second protein mixture contains reduced amount of impurities;
d) applying the second protein mixture to hydrophobic interaction chromatography column;
e) eluting the CTLA-4 IgG1 fusion protein from hydrophobic interaction chromatography column wherein the eluted CTLA-4 IgG1 fusion protein as third protein mixture comprises reduced amount of HMW impurities;
f) applying the third protein mixture to anion exchange chromatography column.
g) eluting the fusion protein from anion exchange chromatography column wherein the eluted fusion protein as fourth protein mixture is substantially free of HMW impurities.

In an embodiment, the invention provides an anion exchange chromatography and hydrophobic interaction chromatography to produce a composition comprising enriched CTLA-4 IgG1 fusion protein and significantly reduced high molecular weight (HMW) impurities selected from about 10%, about 9%, about 8%, about 7%, about 6%, about 5%, about 4%, about 3%, about 2%, about 1%, about 0.5% or less HMW.

In one aspect of this embodiment, the low HMW composition comprises about 0.9% or less HMW, about 0.8% or less HMW, about 0.7% or less HMW, about 0.6% or less HMW, about 0.5% or less HMW, about 0.4% or less HMW, about 0.3% or less HMW, about 0.2% or less HMW, about 0.1% or less HMW.

In an embodiment, the invention provides an anion exchange chromatography and hydrophobic interaction chromatography to produce a composition comprising enriched CTLA-4 IgG1 fusion protein and reduced LMW impurities selected from about 50%, about 40%, about 30%, about 20%, about 10%, about 9%, about 8%, about 7%, about 6%, about 5%, about 4%, about 3%, about 2%, about 1%, about 0.5%, or less LMW.

In an embodiment a method for purifying the fusion protein from a protein mixture comprising fusion protein and pre-peak impurities, the purification process comprises the steps of;
a) obtaining the first fusion protein mixture from the suitable mammalian expression system and the impurities;
b) applying the first fusion protein mixture to affinity chromatography column;
c) eluting the fusion protein mixture from affinity chromatography column as second protein mixture;
d) applying the second fusion protein mixture to hydrophobic interaction chromatography column;
e) eluting the third fusion protein mixture from hydrophobic interaction chromatography column;
f) applying the third fusion protein mixture to anion exchange chromatography column;
g) eluting the fourth fusion protein mixture from anion exchange chromatography column;

wherein the eluted fusion protein is substantially free of pre-peak impurities and reduces 99% or above of pre-peak impurities.

In an embodiment, a method for purifying the fusion protein from a protein mixture comprising fusion protein and high molecular weight (HMW) impurity, the purification process comprises the steps of;
a) obtaining the first fusion protein mixture from the suitable mammalian expression system and the impurities;
b) applying the first fusion protein mixture to affinity chromatography column;
c) eluting the fusion protein mixture from affinity chromatography column as second protein mixture;
d) applying the second fusion protein mixture to hydrophobic interaction chromatography column;
e) eluting the third fusion protein mixture from hydrophobic interaction chromatography column;
f) applying the third fusion protein mixture to anion exchange chromatography column;
g) eluting the fourth fusion protein mixture from anion exchange chromatography column;

wherein the eluted fusion protein is substantially free of HMW impurities and reduces more than 97% of HMW impurities.

In an embodiment, the invention is related to the process for the purification of the fusion protein which is free from at least one impurity selected from Host cell proteins (HCP), Host cell DNA (HCD), Low molecular weight (LMW) Pre-peak and High molecular weight (HMW) impurities comprising:
a) obtaining first protein mixture from the suitable mammalian expression system comprising fusion protein and impurities;
b) applying the first protein mixture to affinity chromatography column;
c) eluting the fusion protein from affinity chromatography column wherein the eluted fusion protein is present in second protein mixture contains reduced amount of said impurities;
d) applying the second protein mixture to hydrophobic interaction chromatography column.
e) eluting the third fusion protein from a hydrophobic interaction chromatography column wherein the eluted fusion protein comprises reduce amount of Pre-peak, HMW and LMW impurities;
f) applying the third protein mixture to anion exchange chromatography column.
g) eluting the fusion protein from anion exchange chromatography column

wherein the eluted fusion protein as fourth protein mixture is substantially free of Pre-peak, HMW and LMW impurities.

In one aspect of such embodiment, the chromatography column selected from anion exchange chromatography and hydrophobic interaction chromatography reduces the high molecular weight impurity to amount below selected from about 20%, about 15%, about 10%, about 5%, about 1%, about 0.9%, about 0.8%, about 0.7%, about 0.6%, about 0.5%, about 0.4%, about 0.3%, about 0.2%, about 0.1% or less.

In one aspect of such embodiment, the chromatography column selected from anion exchange chromatography and hydrophobic interaction chromatography reduces the low molecular weight impurity to amount below selected from about 44%, about 33%, about 23%, about 15%, or less than about 6%, about 5%, about 4%, about 3%, about 2%, about 1%, about 1.5%, about 1.3%, about 1%, about 0.9%, about 0.8%, about 0.7%, about 0.6%, about 0.5%, about 0.4%. In an embodiment, the HIC column reduces impurities more than about 95% or more, about 96% or more, about 97% or more, about 98% or more and about 99% or more.

In an embodiment, the reduction in HMW impurities using protein A chromatography followed by hydrophobic interaction chromatography, optionally followed by ultrafiltration and diafiltration, followed by anion exchange chromatography selected from about 70% or more, about 75% or more, about 80% or more, about 85% or more, about 90% more, about 95% or more, about 96% or more, about 97% or more, about 98% or more.

In an embodiment, the reduction in Pre-peak impurities using protein A chromatography followed by hydrophobic interaction chromatography, optionally followed by ultrafiltration and diafiltration, followed by anion exchange chromatography selected from about 70% or more, about 75% or more, about 80% or more, about 85% or more, about 90% more, about 95% or more, about 96% or more, about 97% or more, about 98% or more.

In an embodiment, the reduction in LMW impurities using protein A chromatography followed by hydrophobic interaction chromatography, optionally followed by ultrafiltration and diafiltration, followed by anion exchange chromatography selected from about 50% or more, about 55% or more, about 60% or more, about 65% or more, about 70% or more, about 75% more, about 80% or more about 80% or more,

In an embodiment, a method for eluting a target protein from a composition comprising one or more impurities selected from pre-peak, low molecular weight (LMW), high molecular weight (HMW) and target protein, wherein a target protein is fusion protein, the purification process comprises the steps of;
a) obtaining the first fusion protein mixture from the suitable mammalian expression system and the impurities;
b) applying the first fusion protein mixture to affinity chromatography column;
c) eluting the fusion protein mixture from affinity chromatography column as second protein mixture;
d) applying the second fusion protein mixture to hydrophobic interaction chromatography column;
e) eluting the third fusion protein mixture from hydrophobic interaction chromatography column;
f) applying the third fusion protein mixture to anion exchange chromatography column;
g) eluting the fourth fusion protein mixture from anion exchange chromatography column;

wherein the eluted fusion protein as fourth protein mixture is substantially free of Pre-peak, HMW and LMW impurities.

In an embodiment, the purification process reduces more than 97% or more of HMW impurities from fusion protein composition.

In an embodiment, the purification process reduces more than 99% or more of Pre-peak impurities from fusion protein composition.

In an embodiment, the purification process reduces more than 80% of LMW impurities from fusion protein composition.

In an embodiment, the fusion protein comprises purity of monomer selected from about 96% or more, about 97% or more, 98% or more and 99% or more.

In an embodiment, the affinity chromatography column resin is selected from Protein A resin, Protein G resin, preferably Protein A resin. Protein A column chromatography resin is selected from Toyopearl AF-rProtein A HC-650, Mab Select Sure LX, MabSelect SuRe, MabSelectXtra, ProSep Ultra Plus, Eshmuno A.

In an embodiment, the first step of affinity chromatography comprises clarified harvest cell culture fluid (HCCF) that is obtained from suitable mammalian expression system. The pH of HCCF is adjusted to pH selected from about pH 8 to about pH 9, preferably pH 7.3±0.2 with 2 M Tris base just before loading onto the affinity column.

In another embodiment, the clarified harvest cell culture fluid (HCCF) can be loaded directly on to the affinity chromatography without any pH adjustment prior.

In an embodiment, prior to loading the Protein A column is equilibrated with a suitable buffer. In an embodiment, the suitable buffer is selected from Tris acetate, Tris-Cl buffer, Sodium Phosphate (NaP), Sodium Chloride (NaCl), HEPES, Triethanolamine, Borate, Glycine-NaOH. In an embodiment, Tris acetate is selected from about 5 mM to about 20 mM and Sodium chloride is selected from about 50 mM to about 200 mM at pH ranging from about pH 6.8 to about pH 7.5 and conductivity is selected from about from 10 mS/cm to about 35 mS/cm, preferably about 16 mS/cm.

In an embodiment, the concentration of equilibration buffer is 20 mM Tris acetate and 150 mM Sodium chloride, pH about 7.3±0.2 and conductivity about 16 mS/cm used to equilibrate the column with at least one column volumes, preferably for 3-5 column volumes.

In an embodiment, the flow rate can be selected from at about 50 cm/hr to at about 300 cm/hr, preferably 300 cm/hr.

In an embodiment, the loading of protein on column, the Protein A column can be washed one or multiple times by using the equilibrating buffer or by employing different buffers. The Protein A column is first washed with the equilibration buffer for at least 3-5 column volumes. This wash can optionally be followed by one or more wash. In another embodiment, the wash buffer is selected from urea, tween 80, isopropanol, NaCl, EDTA, Tris acetate, Sodium Phosphate (NaP), Sodium chloride (NaCl), Tris-HCl, HEPES, Triethanolamine, Borate and Glycine-NaOH. The concentration of wash buffers is selected from 5 mM to about 200 mM and the pH of wash buffer is ranging from pH 2.0 to about pH 7.5 In an embodiment, Protein A column comprises three wash buffers.
a) The first wash buffer comprises Tris Acetate concentration selected from about 5 mM to about 20 mM and concentration selected from about 20 mM to about 150 mM Nacl, at pH 7.3±0.2
b) The second wash buffer comprises Tris Acetate concentration selected from about 5 mM to about 20 mM and NaCl concentration from about 1M to about 10M at pH 7.3±0.2c)
c) The third wash buffer comprises Tris Acetate concentration selected from 5 mM to about 20 mM at pH 7.0±0.2

In an embodiment, the first wash buffer comprises 20 mM Tris Acetate and 150 mM Nacl at pH 7.3±0.2 and conductivity about 16 mS/cm.

In an embodiment, the second wash buffer comprises 20 mM Tris Acetate and 1M NaCl at pH 7.3±0.2 and conductivity about 85 mS/cm.

In an embodiment, the third wash buffer comprises 20 mM Tris Acetateat pH 7.0.±0.2 and conductivity about 1.7 mS/cm.

In an embodiment, the fusion protein from Protein A column can then be eluted using an appropriate suitable buffer. The linear gradient is achieved by using elution buffer selected from pH about 2 to 3.5.

In an embodiment, the elution buffer comprises Tris Acetate concentration selected from about 100 mM to about 200 mM Tris Acetate, preferably 100 mM Tris Acetate at pH 3.5, the conductivity is selected from about from 0.5 mS/cm to about 1.5 mS/cm, preferably about 1 mS/cm.

In an embodiment, the Protein A column further comprises neutralization wash with 20 mM Tris acetate and 150 mM Sodium chloride at pH 7.3±0.2 and conductivity about 16 mS/cm.

In an embodiment, the eluted fractions are collected from about ascending 0.10 AU/cm to descending about 0.30 AU/cm.

In an embodiment, the eluted fractions are collected from ascending 0.25 AU/cm to about descending 0.25 AU/cm.

In an embodiment, the second protein mixture obtained from affinity chromatography column is subjected to suitable treatment to make the protein mixture suitable for loading onto HIC.

In another embodiment, the load preparation done at 1:1 dilution of NPEL (pH 7.5) with 0.8 M Sodium citrate, pH adjusted to 8.0±0.2 before loading.

In an embodiment, the second protein mixture obtained from affinity chromatography column is subjected to a hydrophobic interaction chromatography column and the eluate obtained from HIC column referred as third protein mixture which has reduced level of at least one impurity selected from Host cell proteins (HCP), Host cell DNA (HCD), Low molecular weight (LMW) and High molecular weight (HMW).

In an embodiment, the hydrophobic interaction chromatography resin is selected from Poros Benzyl, Butyl Toyopearl 650 M resin, Toyopearl Phenyl-650, Butyl Sepharose 6 Fast Flow, Butyl Sepharose HP, Phenyl Sepharose 6 Fast Flow high sub, Capto Phenyl high sub, Capto Butyl impRes.

In embodiment, the hydrophobic interaction chromatography is performed in flow through mode. In another embodiment, the hydrophobic interaction chromatography is performed in bind-elute mode.

In an embodiment, the suitable buffer is gradually added in to second protein mixture prior to loading the final conductivity reaches to about from 40 mS/cm to about 70 mS/cm, preferably about 65 mS/cm.

In an embodiment, the suitable buffer is selected from at least one or any combination of the salts selected from Tris acetate, Sodium citrate, Sodium phosphate, Sodium chloride, ammonium sulphate, disodium hydrogen phosphate anhydrous, Trisodium citrate dihydrate, Histidine-HCl, Imidazole, bis-tris, maleate, preferably tris acetate and sodium citrate at pH selected from about pH 6 to about pH 9, preferably pH 8 and conductivity is selected from about from 50 mS/cm to about 80 mS/cm, preferably about 65 mS/cm.

In an embodiment the buffer is selected from Tris acetate, Sodium citrate, disodium hydrogen phosphate anhydrous, Trisodium citrate dihydrate, Histidine-HCl, Imidazole, bis-tris, maleate, Sodium Chloride, Sodium phosphate, Ammonium sulphate.

In an embodiment, the concentration of the buffer is selected from about 10 mM to about 1000 mM. In an embodiment the concentration of buffer is selected from 50 mM to 500 mM. In an embodiment the concentration of buffer is selected from 200 mM to 450 mM.

In an embodiment the concentration of buffer comprises Tris Acetate concentration selected from about 10 mM to about 100 mM.

In an embodiment the concentration of buffer comprises Tris Acetate concentration selected from 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM and 100 mM.

In an embodiment the concentration of buffer comprises Sodium Citrate concentration selected from about 100 mM to about 500 mM.

In an embodiment the concentration of buffer comprises Sodium Citrate concentration selected from 100 mM, 125 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM and 1000 mM.

In an embodiment, the concentration of buffer comprises Sodium Citrate concentration selected from about 0.1M to about 1M.

In an embodiment the concentration of buffer comprises Sodium Citrate concentration selected from 0.1M, 0.2M, 0.3M, 0.4M, 0.5M, 0.6M, 0.7M, 0.8M, 0.9M,1M, 1.2M and 1.5M.

In an embodiment, the equilibration buffer selected from Tris acetate, Tris, Sodium citrate, Sodium chloride (NaCl), Sodium phosphate (NaP), Ammonium sulphate at pH about 6 to about pH 9.

In an embodiment, the equilibration buffer comprises Tris Acetate and Sodium citrate at pH 8±0.2.

In an embodiment the equilibration buffer comprises 50 mM Tris Acetate and 0.4M Sodium citrate at pH8±0.2 and conductivity about 42 mS/cm.

In an embodiment, the load preparation is done with 1:1 dilution of NPEL with 50 mM Tris acetate and 0.8M Sodium citrate at pH 8.0±0.2 and conductivity about 46 mS/cm.

In an embodiment, the wash buffer selected from Tris acetate, Tris, Sodium citrate, Sodium chloride (NaCl), Ammonium sulphate, Sodium phosphate (NaP), at pH about 6 to about pH 9.

In an embodiment, the wash buffer comprises Tris acetate and Sodium citrate at pH 8.0±0.2. In an embodiment the wash buffer comprises 50 mM Tris acetate and 0.4M Sodium citrate at pH 8.0±0.2 and conductivity about 42 mS/cm.

In an embodiment, the elution buffer comprises Tris Acetate concentration selected from 1 mM to about 10 mM.

In an embodiment the elution buffer comprises Tris Acetate concentrated selected from 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM and 20 mM.

In an embodiment, the elution buffer comprises Tris Acetate at pH 8±0.2. In an embodiment the elution buffer comprises 10 mM Tris Acetate at pH 8±0.2 and conductivity about 0.8 mS/cm.

In an embodiment, the process further comprises regeneration buffer. In an embodiment the regeneration buffer is WFI.

In another embodiment the flow rate can be selected from at about 50 cm/hr to at about 400 cm/hr, preferably 300 cm/hr.

In certain embodiment, the gradient was performed for column volume selected from 1CV, 2CV, 3CV, 4CV, 5CV, 6CV, 7CV, 8CV, 9CV, 10CV, 11CV, 12CV, 13CV, 14CV, 15CV, 16CV, 17CV, 18CV, 19CV and 20CV.

In an embodiment, the gradient was performed from 20% B in three column volumes (CV) to 5CV preferably 3CV and 70% B in ten CV to twenty CV preferably fifteen column volume.

In an embodiment, the eluted fractions which are collected from ascending 1.85 AU/cm to about descending 2.0 AU/cm.

In an embodiment, the eluted fractions are collected from ascending 1.0 mAU to about descending 100 mAU.

In an embodiment, the use of hydrophobic interaction chromatography (HIC) process for the purification of fusion protein from mixture comprising protein of interest and High molecular weight (HMW) impurities.

In an embodiment, the protein mixture obtained from hydrophobic interaction chromatography subjected to anion exchange chromatography column and the eluate obtained from hydrophobic interaction chromatography column called as third protein mixture which has reduced level impurities selected from Pre-peak, HMW and LMW.

In another embodiment, the protein mixture obtained from HIC column and optionally after diafiltration can be subjected to an anion exchange chromatography column and the eluate obtained from anion exchange column can be referred as fourth protein mixture which have reduced level of impurities selected from Pre-peak, HMW and LMW.

In an embodiment, the protein is collected in fractions in the Bind and Elute (BT) mode.

In an embodiment, the elute fractions contains protein of interest and substantially free from High molecular weight (HMW) and other impurities.

In an embodiment of the invention is related to the purification of fusion protein from the protein mixture by using hydrophobic interaction chromatography column that can be performed at any step after affinity chromatography column.

In another embodiment, the present invention uses anion exchange chromatography (AEX) column removes impurities selected from Host cell proteins (HCP), Host cell DNA (HCD), Low molecular weight (LMW) and High molecular weight (HMW).

In one embodiment, the anion exchange chromatography is selected from Poros XQ, Poros HQ, DEAE sepharose fast flow, Fractogel® EMD DEAE (M), Toyopearl DEAE-650, Toyopearl DEAE-650, Nuvia Q.

In an embodiment, the anion exchange chromatography is performed in the bind and elute mode.

In an embodiment, the anion exchange chromatography is strong anion exchange chromatography.

In another embodiment, the anion exchange chromatography is performed in flow through mode.

In an embodiment, the loading of the protein mixture from HIC column to anion exchange chromatography column.

In an embodiment, the pH adjusted from about pH 6 to pH 8, preferably 7.5±0.2, prior to loading the column. The conductivity of the sample is adjusted from about 3.0 mS/cm to about 4.2 mS/cm., preferably 3.5 ms/cm.

In an embodiment, the anion exchange chromatography column is equilibrated with suitable buffer selected from histidine hydrochloride, tris acetate, sodium citrate, sodium phosphate, citrate, sodium chloride preferably tris acetate and sodium chloride at pH selected from about 6 to about pH 8, preferably at pH 7.5±0.2, conductivity is selected from about 3.0 mS/cm to about 4.2 mS/cm, preferably 3.5 ms/cm.

In an embodiment, the concentration of the buffer is selected from about 10 mM to about 1000 mM. In an embodiment the concentration of buffer is selected from 50 mM to 500 mM. In an embodiment the concentration of buffer is selected from 200 mM to 450 mM.

In an embodiment the concentration of buffer comprises Tris Acetate concentration selected from about 10 mM to about 100 mM.

In an embodiment the concentration of buffer comprises Tris Acetate concentration selected from 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM and 100 mM.

In an embodiment the concentration of buffer comprises Sodium Chloride concentration selected from about 10 mM to about 50 mM.

In an embodiment the concentration of buffer comprises Sodium Chloride concentration selected from 5 mM, 10 mM, 15 mM, 20 mM, 25 mM, 30 mM, 35 mM, 40 mM, 45 mM, 50 mM, 55 mM and 60 mM.

In an embodiment the concentration of buffer comprises Sodium Chloride concentration selected from about 100 mM to about 1000 mM.

In an embodiment the concentration of buffer comprises Sodium Chloride concentration selected from 100 mM, 200 mM, 300 mM, 400 mM, 500 mM, 600 mM, 700 mM, 800 mM, 900 mM, 1000 mM.

In an embodiment the equilibration buffer comprises Tris Acetate and Sodium Chloride at pH 7.5±0.2. In an embodiment the equilibration buffer comprises 50 mM Tris Acetate and 10 mM Sodium Chloride at pH 7.5±0.2, and conductivity about 3.5 mS/cm.

In an embodiment, the load preparation done with HIC OP was buffer exchanged with AEX EQB and conductivity about 3.5 mS/cm.

In a preferred embodiment, the protein mixture is loaded onto the AEX column. In another embodiment the flow rate can be selected from at about 50 cm/hr to at about 400 cm/hr, preferably 300 cm/hr.

In an embodiment, the wash buffer selected from Tris acetate, Tris, Sodium citrate, Sodium chloride (NaCl), Sodium phosphate (NaP), at pH about 6 to about pH 8 and conductivity is selected from about 2.0 mS/cm to about 12 mS/cm, preferably 3.5 ms/cm.

In an embodiment, the wash buffer comprises Tris acetate and Sodium chloride at pH 7.5±0.2. In an embodiment the wash buffer comprises 50 mM Tris acetate and 10 mM Sodium chloride at pH 7.5±0.2 and conductivity about 3.5 mS/cm.

In an embodiment, the elution buffer selected from Tris acetate, Acetate, Sodium citrate, Sodium chloride (NaCl), Sodium phosphate (NaP), at pH about 6 to about pH 8.

In an embodiment, the elution buffer comprises Tris Acetate concentration selected from 10 mM to about 50 mM. In an embodiment the elution buffer comprises Tris Acetate concentrated selected from 10 mM, 20 mM, 30 mM, 40 mM, 50 mM, 60 mM, 70 mM, 80 mM, 90 mM and 100 mM.

In another embodiment, the elution buffer comprises Sodium Chloride concentration selected from 0.1 M to about 1 M.

In an embodiment, the elution buffer comprises Sodium Chloride concentration selected from 0.1M, 0.2M, 0.3M, 0.4M, 0.5M, 0.6M, 0.7M, 0.8M, 0.9M, 1M, 1.2M and 1.5M.

In an embodiment, the elution is performed with an appropriate buffer. In another embodiment, the elution buffer can be one or mixture of more than one buffer. In an embodiment, the protein is eluted by a 10 mM to 50 mM Tris acetate and 0.1 to 1 M Sodium chloride, preferably 50 mM and 1M at pH 7.5±0.2 and conductivity about 85 mS/cm.

In an embodiment, the gradient was performed from 9% of B to about 30% of B in one to twenty column volumes.

In certain embodiment, the gradient was performed for column volume selected from 1CV, 2CV, 3CV, 4CV, 5CV, 6CV, 7CV, 8CV, 9CV, 10CV, 11CV, 12CV, 13CV, 14CV, 15CV, 16CV, 17CV, 18CV, 19CV and 20CV.

In an embodiment, the process further comprises regeneration buffer.

In an embodiment, the regeneration buffer comprises 50 mM Tris acetate and 1000 mM Sodium chloride at pH about 7.5±0.2 and conductivity about 85 mS/cm.

In an embodiment, the eluted fractions are collected and from ascending 10 mAU to about descending 80 mAU.

In another embodiment, the use of anion exchange chromatography (AEX) process for the purification of fusion protein from mixture comprising protein of interest and reduced impurities selected from Low molecular weight (LMW) and High molecular weight (HMW) impurities.

In an embodiment, the reduction in pre-peak impurity selected from about 25%, about 24%, about 23%, about 22%, about 21%, about 20%, about 19%, about 18%, about 17%, about 16%, about 15%, about 14%, about 13%, about 12%, about 11%, about 10%, about 5%, about 3%, about 1%, or below quantifiable limit.

In an embodiment, the elute fractions contain substantially purified protein of interest and reduced Host cell proteins (HCP), Host cell DNA (HCD) and Low molecular weight (LMW),Pre-peak and High molecular weight (HMW) impurities.

In an embodiment, the elute is collected and can be referred as final protein of interest.

In an embodiment, the protein mixture obtained from AEX column subjected to ultra-filtration and diafiltration with reduced level of impurities.

In one aspect of such embodiment, the filtrate is substantially free from viruses, adventitious agents. LMW, HMW and pre-peak.

### Example 1—Purification of Fc-Fusion Protein by Performing Affinity Chromatography Followed by HIC (Hydrophobic Interaction Chromatography) and AEX (Anion Exchange Chromatography)

All chromatographic processes were carried out using an AKTA Pure 150 system from Cytiva (formally known as GE Healthcare). Concentration of protein samples were determined by measuring absorbance at 280 nm using Shimadzu Spectrophotometer. Protein A Chromatography resin (Mab Select Sure LX) obtained from Cytiva, HIC (Poros Benzyl) resins were obtained from Thermofisher, AEX (Poros XQ) and Column hardwares of VL22/250 and VL16/250 were obtained from Millipore, whereas XK16/40 and Hiscale 10/40 column was obtained from GE Healthcare. 30 kDa Pellicone 3 cassette and 10 kDa Amicon were obtained from Millipore. 200 cm² TFF cassette holder obtained from sartorius and refrigerated centrifuge obtained from Beckman coulter. All GMP grade chemicals were obtained from J. T.Baker or Merck Millipore.

A Fc-fusion protein expressed in Chinese Hamster Ovary (CHO) cell line is captured using Protein A resin (MabSelectSure LX, GE Healthcare) packed in VL 22/250 column. Eluted protein is further purified using HIC chromatography (Poros Benzyl, Thermofisher) packed in VL16/250. Final polishing was performed by AEX chromatography (Poros XQ, Thermofisher) packed in Hiscale 10/40 column.

A method of purifying a Fc-fusion protein from one or more impurities in a sample, comprising the steps of:
a) obtaining first protein mixture from the suitable mammalian expression system comprising Fc-fusion protein,
b) binding the Fc-fusion protein present in the sample to a Protein A affinity chromatography resin and eluting the Fc-fusion protein from the Protein A resin, for which experiment design is showing in table 1, wherein the eluted product was neutralized and provides a second sample, optionally referred to as a Neutralized Protein A Elute (NPEL). The residence time is 4 min for all the phases.
c) Neutralized protein A eluate (NPEL) was loaded on HIC resin and elution were performed with a low conductivity buffer, wherein the eluted product provides a third sample, for which experiment design is showing table 2, optionally referred to as a hydrophobic interaction chromatography output (HIC OP). The residence time is 4 min for all the phases.
d) HIC OP was further concentrated, and buffer exchanged in a suitable buffer with Pellicon 3 cassette with D-screen (176 cm²), provides protein sample for which experiment design is showing in Table 3, optionally referred as ultrafiltration/diafiltration-I output (UFDF-I OP).
e) binding the UFDF-J OP to an anion exchange chromatography resin (AEX) and eluting the fourth sample from the AEX resin, wherein the eluted product provides a sample for which experiment design is shown in table 4, optionally referred to as an anion exchange chromatography output (AEX OP) the SE-HPLC analysis (for Pre-peak and HMW) and CE-SDS (for LMW) of protein sample is shown in table 5 and table 6.

TABLE 1
Experiment Design for Affinity chromatography:

| | | Residence | Column |
|---|---|---|---|
| | | Time | Volume |
| Step | Buffer | (min) | (CV) |
| Equili- | 20 mM TrisAc + 150 mM NaCl, | 4 | 3-5 |
| bration | pH 7.3 ± 0.2 | | |
| Load | Clarified Harvest | 4 | As per load |
| | | | density |
| Wash 1 | 20 mM Tris Acetate + 150 mM | 4 | 3-5 |
| | Nacl, pH 7.3 | | |
| Wash 2 | 20 mM Tris Acetate + 1 M | 4 | 3-5 |
| | Nacl, pH 7.3 | | |
| Wash 3 | 20 mM Tris Acetate, pH 7.0 | 4 | 5-7 |
| Elution | 100 mM Acetate, pH 3.5 | 4 | 3-5 |
| Neutral- | 20 mM Tris Acetate + 150 mM | 4 | 3-5 |
| ization | Nacl, pH 7.3 | | |
| wash | | | |
| CIP | 0.1M NaOH | 4 | 3-5 |
| WFI | WFI | 4 | 3-5 |
| Storage | 2% Benzyl alcohol in wash 3 | 4 | 3-5 |
| | buffer | | |

TABLE 2
Experiment design for Hydrophobic interaction chromatography

| | | Residence | Column |
|---|---|---|---|
| | | Time | Volume |
| Step | Buffer | (min) | (CV) |
| Stock buffer | 50 mM TrisAc + 0.8M Sodium | NA | NA |
| | Citrate, pH 8.0 ± 0.2 | | |
| Equilibration | 50 mM TrisAc + 0.4M Sodium | 4 | 4-6 |
| | Citrate, pH 8.0 ± 0.2 | | |
| Load | 1:1 dilution of NPEL with stock | 4 | As per load |
| | buffer | | density |
| Wash | 50 mM TrisAc + 0.4M Sodium | 4 | 3-5 |
| | Citrate, pH 8.0 ± 0.2 | | |
| Elution | 10 mM TrisAc, pH 8.0 ± 0.2 | 4 | 20% B for |
| | | | 3 CV |
| | | | 70% B in |
| | | | 15 CV |
| Regeneration | WFI | | 4-6 |
| CIP | 1.0M NaOH solution | 4 | 3-5 |
| Storage | 0.1M NaOH solution | 4 | 3-5 |

TABLE 3
Experiment design for UFDF-I

| Parameter/Step | Value |
|---|---|
| Cross Flow | 360 LMH |
| Membrane Area | 176 cm2 |
| TMP | <1 bar |
| Membrane | 0.5 L of 0.5M Sodium hydroxide solution |
| Sanitization | |
| Membrane | 50 mM TrisAc + 0.2M Sodium Citrate + 0.1M |
| Equilibration | Arginine, pH 8.0 ± 0.2 |
| Ultrafiltration | Concentrate AEX OP to 6-9 times of starting volume |
| and Diafiltration | Buffer exchange the concentrated HIC OP in to |
| | 50 mM TrisAc + 10 mM sodium chloride, |
| | pH 7.5 ± 0.2, Total DV: 6-8 DV |
| Sanitization | 0.5N Sodium Hydroxide (0.5 L) |
| Storage | 0.1N Sodium Hydroxide (0.2 L) |

TABLE 4
Experiment design for Anion Exchange chromatography

| | | Residence | Column |
|---|---|---|---|
| | | Time | Volume |
| Step | Buffer | (min) | (CV) |
| Equili- | 50 mM TrisAc + 10 mM sodium | 4 | 3-5 |
| bration | chloride, pH 7.5 ± 0.2 | | |
| Load | Buffer exchanged HIC OP | 4 | As per load |
| | | | density |
| Wash | 50 mM TrisAc + 10 mM sodium | 4 | 3-5 |
| | chloride, pH 7.5 ± 0.2 | | |
| Elution | 50 mM TrisAc + 1M sodium | 4 | 9% B for |
| | chloride, pH 7.5 ± 0.2 | | 3 CV |
| | | | 30% B in |
| | | | 20 CV |
| Regen- | 50 mM TrisA + 1000 mM sodium | 4 | 3-5 |
| eration | chloride, pH 7.5 ± 0.2 | | |
| CIP | 0.5M NaOH solution | 4 | 3-5 |
| Storage | 0.1M NaOH solution | 4 | 3-5 |

TABLE 5
SE-HPLC Analysis of all samples

| | | | % Pre- |
|---|---|---|---|
| Process Step output | % Monomer | % HMW | peak |
| NPEL | 70.60 | 15.18 | 14.15 |
| HIC OP >> UFDF I OP | 98.05 | 0.20 | 0 |
| AEX OP | 99.65 | 0.35 | 0 |

It is evident from the data that composition obtained from above mentioned three chromatographic steps purification that shows substantially reduced HMW and Pre-peak impurities purity of protein 99.65% analysed by SE HPLC analysis.

TABLE 6
Size related Variants: CE-SDS

| Process Step output | % Monomer |
|---|---|
| NPEL | 72.08 |
| HIC OP >> UFDF-I OP | 82.58 |
| AEX OP | 93.81 |

It is evident from the data that composition obtained from above mentioned three chromatographic steps purification that shows improvement of monomer purity measured by CE SDS analysis.

1. A method for purifying a fusion protein from a protein mixture comprising a fusion protein and high molecular weight (HMW) impurity, the purification process comprises the steps of;
a) obtaining the first fusion protein mixture from the suitable mammalian expression system and the impurities;
b) applying the first fusion protein mixture to affinity chromatography column;
c) eluting the fusion protein mixture from affinity chromatography column as second protein mixture;
d) applying the second fusion protein mixture to hydrophobic interaction chromatography column;
e) eluting the third fusion protein mixture from hydrophobic interaction chromatography column;
f) applying the third fusion protein mixture to anion exchange chromatography column;
g) eluting the fourth fusion protein mixture from anion exchange chromatography column;
wherein the eluted fusion protein is substantially free of HMW impurities and reduces more than 97% of HMW impurities.

2. A method for purifying a fusion protein from a protein mixture comprising a fusion protein and pre-peak impurities the purification process comprises the steps of;
a) obtaining the first fusion protein mixture from the suitable mammalian expression system and the impurities;
b) applying the first fusion protein mixture to affinity chromatography column;
c) eluting the fusion protein mixture from affinity chromatography column as second protein mixture;
d) applying the second fusion protein mixture to hydrophobic interaction chromatography column;
e) eluting the third fusion protein mixture from hydrophobic interaction chromatography column;
f) applying the third fusion protein mixture to anion exchange chromatography column;
g) eluting the fourth fusion protein mixture from anion exchange chromatography column;
wherein the eluted fusion protein is substantially free of pre-peak impurities and reduces 99% or above of pre-peak impurities.

3. The process as claimed in claim 1, wherein the eluted fusion protein has more than 99% purity of monomer.

4. The process as claimed in claim 1, wherein the Hydrophobic interaction chromatography and anion exchange chromatography is performed in bind-elute mode.

5. The process as claimed in claim 1, wherein the second protein mixture is loaded onto hydrophobic interaction chromatography (HIC) at suitable pH selected from about 6 to about 9, preferably pH 8.0±0.2 and suitable conductivity selected from about 40 mS/cm to about 70 mS/cm.

6. The process as claimed in claim 5, wherein the suitable pH and/or conductivity is maintained with suitable buffer selected from Tris acetate, Sodium citrate, Acetate, Sodium chloride.

7. The process as claimed in claim 1, wherein the elution from hydrophobic interaction chromatography is performed at suitable pH 8.0±0.2 with buffer selected from Tris acetate, Sodium citrate, Acetate, Sodium chloride.

8. The process as claimed in claim 1, wherein the hydrophobic interaction chromatography resin is selected from Poros Benzyl, Butyl Toyopearl 650 M resin, Toyopearl Phenyl-650, Butyl Sepharose 6 Fast Flow, Phenyl Sepharose 6 Fast Flow, Butyl Sepharose HP, Phenyl Sepharose 6 Fast Flow high sub, Capto Phenyl high sub, and Capto Butyl impRes.

9. The process as claimed in claim 1, wherein the Affinity chromatography is selected from Protein A, Protein G and Protein L.

10. The process as claimed in claim 1, wherein the anion exchange resin is selected from Poros XQ, Poros HQ DEAE, Sepharose fast flow, Fractogel® EMD DEAE (M), Toyopearl DEAE-650, Toyopearl DEAE-650, and Nuvia Q.

11. The process as claimed in claim 1, wherein fusion protein mixture is loaded on to anion exchange chromatography at suitable pH selected from about 7 to about 8, preferably 7.5±0.2 maintained with suitable buffer selected from Tris acetate, sodium citrate, and Sodium chloride.

12. The process as claimed in claim 1, wherein the elution is performed from anion exchange chromatography with conductivity selected from 40 mS/cm to 90 mS/cm, preferably 85 mS/cm in linear gradient by increasing to elution buffer by about 9% or about 20% or about 25% or about 30%.

13. The process as claimed in claim 12, wherein the eluted fusion protein is collected from ascending 10 mAU to about descending 80 mAU.

14. The process as claimed in claim 1, wherein fusion protein is selected from CTLA4-IgG1, TNFR-IgG1, VEGF-IgG1.

15. A pharmaceutical composition of CTLA4-IgG1 or CTLA4-Fc fusion protein comprising:
(a) substantially purified monomer of said fusion protein having purity at least 90% and HMW less than about 0.3%, measured by SE-HPLC; or
(b) substantially purified monomer of said fusion protein having purity at least 90% and pre-peak is less than about 0.1%, measured by SE-HPLC.

16. (canceled)

17. The process as claimed in claim 2, wherein the eluted fusion protein has more than 99% purity of monomer.

18. The process as claimed in claim 2, wherein the elution from hydrophobic interaction chromatography is performed at suitable pH 8.0±0.2 with buffer selected from Tris acetate, Sodium citrate, Acetate, Sodium chloride.

19. The process as claimed in claim 2, wherein the hydrophobic interaction chromatography resin is selected from Poros Benzyl, Butyl Toyopearl 650 M resin, Toyopearl Phenyl-650, Butyl Sepharose 6 Fast Flow, Phenyl Sepharose 6 Fast Flow, Butyl Sepharose HP, Phenyl Sepharose 6 Fast Flow high sub, Capto Phenyl high sub, and Capto Butyl impRes.

20. The process as claimed in claim 2, wherein the anion exchange resin is selected from Poros XQ, Poros HQ DEAE, Sepharose fast flow, Fractogel® EMD DEAE (M), Toyopearl DEAE-650, Toyopearl DEAE-650, and Nuvia Q.

21. The process as claimed in claim 2, wherein fusion protein mixture is loaded on to anion exchange chromatography at suitable pH selected from about 7 to about 8, preferably 7.5±0.2 maintained with suitable buffer selected from Tris acetate, sodium citrate, and Sodium chloride.

22. The process as claimed in claim 2, wherein the elution is performed from anion exchange chromatography with conductivity selected from 40 mS/cm to 90 mS/cm, preferably 85 mS/cm in linear gradient by increasing to elution buffer by about 9% or about 20% or about 25% or about 30%.
